(19) United States
(12) Patent Application Publication
(10) Pub. No.: US 2021/0039958 A1
(43) Pub. Date: Feb. 11, 2021

(54) TITANIUM DIOXIDE PARTICLES

(71) Applicant: Croda International Plc, Goole, East Yorkshire (GB)

(72) Inventors: Ian Robert Tooley, Goole, East Yorkshire (GB); Robert Michael Sayer, Goole, East Yorkshire (GB); Amy Rose Goddard, Goole, East Yorkshire (GB)

(73) Assignee: Croda International Plc, Goole, East Yorkshire (GB)

(21) Appl. No.: 17/044,670

(22) PCT Filed: Apr. 11, 2019
(86) PCT No.: PCT/EP2019/059345

§ 371 Date: Oct. 1, 2020

(30) Foreign Application Priority Data
Apr. 12, 2018 (GB) 1806041.8

(51) Int. Cl. C01G 23/08; A61Q 17/04; A61K 8/29

(52) U.S. Cl. CPC C01G 23/08; A61Q 17/04; A61K 8/29; A61K 2800/413; C01P 2006/12; C01P 2006/16; C01P 2002/60; C01P 2004/64

(57) ABSTRACT

Titanium dioxide particles have high UVB absorption properties, an effective UVA efficacy and transparency. The titanium dioxide can be produced by calcining precursor titanium dioxide particles. The titanium dioxide particles can be used to form dispersions. The titanium dioxide particles and dispersions thereof can be used to produce sunscreen products which are suitable for use in a wide range of personal care applications.

### FIELD OF INVENTION

The present invention relates to titanium dioxide particles and a method of making thereof, a dispersion made therefrom, and in particular to the use thereof in a personal care product.

### BACKGROUND

Titanium dioxide has been employed as an attenuator of ultraviolet light in a wide range of applications such as sunscreens, organic resins, films and coatings.

It is well known that both UVA and UVB radiation can play an important role in premature skin ageing and skin cancer. Thus, protection against both UVA and UVB radiation is vitally important to the end-user. There is a constant need to improve the balance of properties of inorganic, particularly titanium dioxide, sunscreens. This is particularly so because the demand for “inorganic only” sunscreens has increased in recent years, due to concerns over the toxicity of various organic UV absorbers, and the “yellowing” impact some organic UV absorbers have on inorganic sunscreens.

Thus, there is a requirement for a particulate titanium dioxide which exhibits high UVB absorption properties, but also has an effective UVA efficacy and acceptable transparency, particularly in a non-nanoparticulate form, which can be used in a wide range of applications

### SUMMARY OF THE INVENTION

We have now surprisingly discovered an improved titanium dioxide, and method of making thereof, which overcomes or significantly reduces at least one of the aforementioned problems.

Accordingly, the present invention provides titanium dioxide particles comprising a volume based median particle diameter D(v,0.5) of greater than 175 nm and an (E₃₀₈×E360)/E524 value of greater than 300 l/g/cm.

The invention also provides titanium dioxide particles comprising (i) a mean crystal size of 30.0 to 51.0 nm, and/or (ii) a mean aspect ratio of 1.05 to 1.55:1.

The invention further provides titanium dioxide particles comprising (i) an (E308×E360)/E524 value of greater than or equal to 320 l/g/cm, and optionally (ii) an E524 of less than or equal to 7.5 l/g/cm, and/or an E₃₀₈×E360 value of greater than 2100 (l/g/cm)².

The invention also further provides a dispersion comprising a dispersing medium and titanium dioxide particles as defined herein.

The invention yet further provides a sunscreen product comprising titanium dioxide particles and/or a dispersion thereof, as defined herein.

The invention even further provides a method of producing titanium dioxide particles which comprises (i) forming precursor titanium dioxide particles having a mean aspect ratio of 3.0 to 7.0:1, (ii) calcining the precursor particles to produce calcined titanium dioxide particles having a mean crystal size of 30.0 to 51.0 nm and/or a mean aspect ratio of 1.05 to 1.55:1, and optionally (iii) applying an inorganic and/or organic coating to the calcined titanium dioxide particles.

The invention still further provides a method of heating precursor titanium dioxide particles at a temperature of greater than 400° C. to produce calcined titanium dioxide particles wherein (i) the mean width of the titanium dioxide particles is increased by 60 to 200%, and/or (ii) the BET specific surface area is reduced by 35 to 95%, and/or (iii) the mean crystal size is increased by 200 to 400%.

The invention yet even further provides titanium dioxide particles obtainable by a process which comprises (i) forming precursor titanium dioxide particles having a mean aspect ratio of 3.0 to 7.0:1, (ii) calcining the precursor particles to produce calcined titanium dioxide particles, and optionally (iii) applying an inorganic and/or organic coating to the calcined titanium dioxide particles, wherein the titanium dioxide particles have an E₅₂₄ of less than or equal to 7.5 l/g/cm and an (E₃₀₈×E₃₆₀)/E₅₂₄ value of greater than or equal 320 l/g/cm.

The invention also even further provides the use of calcination to improve the UV absorption properties of titanium dioxide particles wherein the calcined particles comprise an (E₃₀₈×E₃₆₀)/E₅₂₄ value of greater than or equal to 320 l/g/cm.

The titanium dioxide particles according to the present invention preferably comprise anatase and/or rutile crystal form. The titanium dioxide in the particles suitably comprises a major portion of rutile, preferably greater than 70%, more preferably greater than 80%, particularly greater than 90%, and especially greater than 95% and up to 100% by weight of rutile.

The particles may be prepared by standard procedures, such as using the chloride process, or by the sulphate process, or by the hydrolysis of an appropriate titanium compound such as titanium oxydichloride or an organic or inorganic titanate, or by oxidation of an oxidisable titanium compound, e.g. in the vapour state.

In one embodiment, the titanium dioxide particles may be doped with a dopant metal selected from the group consisting of aluminium, chromium, cobalt, copper, gallium, iron, lead, manganese, nickel, silver, tin, vanadium, zinc, zirconium, and combinations thereof. The dopant is preferably selected from the group consisting of chromium, cobalt, copper, iron, manganese, nickel, silver, and vanadium, more preferably from manganese and vanadium, particularly manganese, and especially in the 2+ and/or 3+ state.

Doping can be performed by normal methods known in the art. Doping is preferably achieved by co-precipitation of titanium dioxide and a soluble dopant complex such as manganese chloride or manganese acetate. Alternatively, doping can be performed by a baking technique by heating a titanium complex in the presence of a dopant complex, e.g. manganese nitrate, at a temperature of greater than 500° C. and normally up to 1,000° C. Dopants can also be added by oxidizing a mixture containing a titanium complex and dopant complex, e.g. manganese acetate, such as by spraying the mixture through a spray atomizer into an oxidation chamber.

Doped titanium dioxide particles preferably comprise in the range from 0.01 to 3%, more preferably 0.05 to 2%, particularly 0.1 to 1%, and especially 0.5 to 0.7% by weight of dopant metal, preferably manganese, based on the weight of titanium dioxide.

In one embodiment, initial or precursor titanium dioxide particles are prepared, for example, by the hydrolysis of a titanium compound, particularly of titanium oxydichloride, and these precursor particles are then subjected to a calcination process in order to obtain titanium dioxide particles according to the present invention.

The precursor titanium dioxide particles preferably comprise a rutile content as hereinbefore described. In addition, the precursor titanium dioxide particles preferably comprise less than 10%, more preferably less than 5%, and particularly less than 2% by weight of amorphous titanium dioxide. The remaining titanium dioxide (i.e. up to 100%) is in crystalline form. In one embodiment, the titanium dioxide in the precursor particles preferably is substantially all in crystalline form.

The individual precursor titanium dioxide particles are suitably acicular in shape and have a long axis (maximum dimension or length) and short axis (minimum dimension or width). The third axis of the particles (or depth) is preferably approximately the same dimensions as the width.

The mean length by number of the precursor titanium dioxide particles is suitably in the range from 40.0 to 85.0 nm, preferably 45.0 to 80.0 nm, more preferably 50.0 to 75.0 nm, particularly 55.0 to 70.0 nm, and especially 60.0 to 65.0 nm. The mean width by number of the particles is suitably in the range from 8.0 to 22.0 nm, preferably 10.0 to 20.0 nm, more preferably 12.0 to 18.0 nm, particularly 13.0 to 17.0 nm, and especially 14.0 to 16.0 nm. The precursor titanium dioxide particles suitably have a mean aspect ratio d₁:d₂ (where d₁ and d₂, respectively, are the length and width of the particle) in the range from 3.0 to 7.0:1, preferably 3.5 to 6.5:1, more preferably 4.0 to 6.0:1, particularly 4.5 to 5.5:1, and especially 4.8 to 5.2:1. The size of the precursor particles can be determined, as herein described, by measuring the length and width of particles selected from a photographic image obtained by using a transmission electron microscope.

The precursor titanium dioxide particles may have a mean crystal size (measured by X-ray diffraction as herein described) in the range from 6.0 to 15.0, suitably 7.0 to 13.5 nm, preferably 8.0 to 12.5 nm, more preferably 9.0 to 11.5 nm, particularly 9.5 to 10.5 nm, and especially 9.8 to 10.2 nm.

The size distribution of the crystal size of the precursor titanium dioxide particles can be important, and suitably at least 40%, preferably at least 50%, more preferably at least 60%, particularly at least 70%, and especially at least 80% by weight of the titanium dioxide particles have a crystal size within one or more of the above preferred ranges for the mean crystal size.

The precursor titanium dioxide particles may have a BET specific surface area, measured as herein described, in the range from 75 to 140, suitably 80 to 125, preferably 87 to 115, more preferably 92 to 110, particularly 97 to 105, and especially 99 to 103 m²g⁻¹.

The precursor titanium dioxide particles may have (i) an average pore diameter, measured as herein described by mercury porosimetry, in the range from 40 to 115, suitably 50 to 105, preferably 60 to 95, more preferably 65 to 90, particularly 70 to 85, and especially 75 to 80 nm; and/or (ii) a total pore area at 59,950.54 psia, measured as herein described by mercury porosimetry, in the range from 35 to 105, suitably 45 to 95, preferably 55 to 85, more preferably 63 to 80, particularly 68 to 77, and especially 71 to 74 m²g⁻¹.

In one embodiment, the precursor titanium dioxide particles herein described are preferably calcined for less than 2 hours, more preferably for 2 minutes to 1.5 hours, particularly for 5 minutes to 1 hour, and especially for 10 to 30 minutes. The precursor titanium dioxide particles may be calcined at a temperature of greater than 400° C., suitably in the range from 450 to 900° C., preferably 500 to 850° C., more preferably 550 to 800° C., particularly 600 to 750° C., and especially 650 to 720° C.

For plant-scale production, e.g. for quantities greater than 50 Kg/hour, the precursor titanium dioxide particles are suitably calcined at a temperature in the range from 500 to 850° C., preferably 650 to 770° C., more preferably 690 to 730° C., particularly 700 to 720° C., and especially 705 to 715° C.

In one embodiment, a continuous calcining process is employed wherein the precursor titanium dioxide particles are passed through a rotating calciner which is preferably heated indirectly. A drum preferably rotates as it is heated and the velocity of the trommel determines the retention time of the titanium dioxide particles in the oven. The velocity of the trommel is preferably in the range from 500 to 1,000, more preferably 600 to 900 r.p.m. The feeding rate of titanium dioxide particles into the oven can be operated continuously, suitably by a screw conveyor, preferably in the range from 5 to 50%, more preferably 10 to 40%, particularly 15 to 30%, and especially approximately 25% by weight of the total capacity of the screw conveyor. The feeding rate of the titanium dioxide into the oven, e.g. for plant-scale production, is preferably in the range from 50 to 150 Kg/hour, more preferably 70 to 130 Kg/hour, particularly 90 to 110 Kg/hour, and especially 95 to 105 Kg/hour.

In one embodiment, a pre-drying stage is not used and the precursor titanium dioxide particles subjected to the calcination process may comprise in the range from 40 to 75%, preferably 50 to 70%, more preferably 55 to 65%, and particularly about 60% by weight of water based on the total weight of the particles.

In another embodiment, a pre-drying stage is employed, e.g. by heating the precursor titanium dioxide particles, preferably on a fluid bed, at approximately around 150° C. for about 2 hours. The dried precursor titanium dioxide particles subjected to the calcination process preferably comprise in the range from 1 to 15%, more preferably 4 to 10%, particularly 5 to 7%, and especially 5.5 to 6.5% by weight of water based on the total weight of the particles.

The calcined titanium dioxide particles may have a BET specific surface area, measured as herein described, of greater than or equal to 24, suitably in the range from 24 to 42, more suitably 27 to 39, preferably 29 to 37, more preferably 30 to 36, particularly 31 to 35, and especially 32 to 34 m²g⁻¹.

In one embodiment, the calcination process herein described results in a reduction in the BET specific surface area of the titanium dioxide particles (from precursor to calcined), suitably by an amount in the range from 35 to 95%, suitably 45 to 85%, preferably 55 to 80%, more preferably 60 to 75%, particularly 64 to 70%, and especially 66 to 68% based on the BET specific surface area of the precursor particles.

The calcined titanium dioxide particles may have (i) an average pore diameter, measured as herein described by mercury porosimetry, in the range from 75 to 160, suitably 85 to 150, preferably 95 to 140, more preferably 105 to 130, particularly 110 to 125, and especially 115 to 120 nm; and/or (ii) a total pore area at 59,950.54 psia, measured as herein described by mercury porosimetry, in the range from 20 to 53, suitably 24 to 48, preferably 28 to 44, more preferably 31 to 41, particularly 33 to 39, and especially 35 to 37 m²g⁻¹.

In one embodiment, the calcination process herein described results in (i) a reduction in the total pore area at 59,950.54 psia of the titanium dioxide particles (from precursor to calcined), measured as herein described by mercury porosimetry, by an amount in the range from 20 to 80%, suitably 30 to 70%, preferably 40 to 60%, more preferably 45 to 56%, particularly 48 to 53%, and especially 50 to 51% based on the total pore area at 59,950.54 psia of the precursor particles; and/or (ii) an increase in the average pore diameter of the titanium dioxide particles (from precursor to calcined), measured as herein described by mercury porosimetry, by an amount in the range from 10 to 90%, suitably 20 to 70%, preferably 30 to 55%, more preferably 35 to 47%, particularly 38 to 44%, and especially 40 to 42% based on the average pore diameter of the precursor particles.

The calcined titanium dioxide particles suitably have a mean aspect ratio d₁:d₂ (where d₁ and d₂, respectively, are the length and width of the particle) in the range from 1.05 to 1.55:1, preferably 1.10 to 1.50:1, more preferably 1.15 to 1.45:1, particularly 1.20 to 1.40:1, and especially 1.25 to 1.35:1. The third axis of the particles (or depth) is preferably approximately the same dimensions as the width.

The mean length by number of the titanium dioxide particles is suitably in the range from 32.0 to 56.0 nm, preferably 37.0 to 51.0 nm, more preferably 40.0 to 48.0 nm, particularly 42.0 to 46.0 nm, and especially 43.0 to 45.0 nm. The mean width by number of the particles is suitably in the range from 22.0 to 46.0 nm, preferably 27.0 to 41.0 nm, more preferably 30.0 to 38.0 nm, particularly 32.0 to 36.0 nm, and especially 33.0 to 35.0 nm. The size of the titanium dioxide particles can be determined, as herein described, by measuring the length and width of particles selected from a photographic image obtained by using a transmission electron microscope.

In one embodiment, the calcination process herein described results in an increase in the mean width by number of the titanium dioxide particles (from precursor to calcined), suitably by an amount in the range from 60 to 200%, preferably 80 to 180%, more preferably 95 to 160%, particularly 110 to 145%, and especially 120 to 135% based on the mean width by number of the precursor particles.

The calcined titanium dioxide particles may have a mean crystal size (measured by X-ray diffraction as herein described) in the range from 30.0 to 51.0 nm, suitably 34.0 to 51.0 nm, preferably 37.0 to 47.0 nm, more preferably 39.0 to 44.0 nm, particularly 41.0 to 44.0 nm, and especially 42.0 to 43.0 nm.

The size distribution of the crystal size of the calcined titanium dioxide particles can be important, and suitably at least 50%, preferably at least 60%, more preferably at least 70%, particularly at least 80%, and especially at least 90% by weight of the titanium dioxide particles have a crystal size within one or more of the above preferred ranges for the mean crystal size.

In one embodiment, the calcination process herein described results in an increase in the mean crystal size of the titanium dioxide particles (from precursor to calcined), suitably by an amount in the range from 200 to 400%, preferably 235 to 375%, more preferably 260 to 350%, particularly 280 to 330%, and especially 295 to 315% based on the mean crystal size of the precursor particles.

In one embodiment of the present invention, the titanium dioxide, preferably calcined, particles according to the invention are coated with an inorganic and/or organic coating. Doped titanium dioxide particles may be uncoated, i.e. consist essentially of titanium dioxide and dopant.

In one embodiment the inorganic coating is an oxide of aluminium, zirconium or silicon, or mixtures thereof such as alumina and silica. The amount of inorganic coating, preferably alumina and/or silica, is suitably in the range from 1 to 12%, preferably 2 to 6%, to more preferably 2.5 to 4.5%, particularly 3 to 4%, and especially 3.3 to 3.7% by weight, based on the weight of titanium dioxide core (or uncoated) particles.

In one embodiment of the invention, the titanium dioxide particles are hydrophobic. The hydrophobicity of the titanium dioxide can be determined by pressing a disc of titanium dioxide powder, and measuring the contact angle of a drop of water placed thereon, by standard techniques known in the art. The contact angle of a hydrophobic titanium dioxide is preferably greater than 50°.

The titanium dioxide particles can be coated with a hydrophobizing agent in order to render them hydrophobic. Suitable coating materials are water-repellent, preferably organic, and include fatty acids, preferably fatty acids containing 10 to 20 carbon atoms, such as lauric acid, stearic acid and isostearic acid, salts of the above fatty acids such as sodium, potassium and/or aluminium salts, fatty alcohols, such as stearyl alcohol, and silicones such as polydimethylsiloxane and substituted polydimethylsiloxanes, and reactive silicones such as methylhydrosiloxane and polymers and copolymers thereof. Stearic acid and/or salt thereof is particularly preferred.

In one embodiment, the titanium dioxide particles are treated with up to 15%, suitably in the range from 1 to 10%, preferably 2.5 to 7.5%, more preferably 3.5 to 6%, particularly 4 to 5.2%, and especially 4.4 to 4.8% by weight of fatty acid, based on the weight of the titanium dioxide core particles.

In one embodiment, the coating layer comprises a silane coupling agent, preferably an organosilane, and more preferably of general Formula (1);

X₄₋ₙ—Si-[Lₘ-Y]ₙ (1)

wherein

Y is a functional group,

X is a hydrolysable group,

L is a linking group,

m is 0 or 1, preferably 1, and

n is 1 or 2, preferably 1.

Thus, a preferred silane coupling agent is of the general formula X₃—Si-L-Y. The at least one functional group (Y) may be, for example, selected from the group consisting of methyl, ethyl, vinyl, carboxyl, glycidoxy, epoxy, glycidyl, amino, mercapto, acrylic, and methacrylic group. The functional group preferably comprises a nitrogen atom, and more preferably is an amine group. The amine group may be a primary, secondary, tertiary or quaternary group, and is preferably a primary amine group.

The preferred amine group is suitably of formula —NR₂, wherein each R individually is, or comprises, a group selected from the group consisting of hydrogen, lower (i.e. C1-C6) alkyl, aryl, lower alkylaryl, lower arylalkyl, alkenyl, cycloalkenyl, alkene, alkylene, arylene, alkylarylene, arylalkylene and cycloalkylene. In a preferred embodiment, each R is individually selected from the group consisting of hydrogen and a linear or branched C1-C6 alkyl group, more preferably hydrogen and a C1-C4 alkyl group, and particularly where both R groups are hydrogen.

The at least one hydrolysable group (X) may be —OR¹, —Cl, —Br, —I, and preferably is —OR¹, wherein each R′ individually is, or comprises, a group selected from the group consisting of hydrogen, lower (i.e. C1-C6) alkyl, aryl, lower alkylaryl, lower arylalkyl, alkenyl, cycloalkenyl, alkene, alkylene, arylene, alkylarylene, arylalkylene and cycloalkylene. Preferably each R¹ is individually selected from the group consisting of hydrogen and a linear or branched C1-C6 alkyl group, more preferably a C1-C4 alkyl, particularly a C1-C2 alkyl group, and especially an ethyl group.

The optional linking group (L) may comprise or consist of an alkyl, aryl, alkylaryl, arylalkyl, cycloalkyl, alkenyl, cycloalkenyl, alkene, alkenylene, cycloalkenylene, alkylene, arylene, alkylarylene, arylalkylene, and/or cycloalkylene group. The linking group is preferably a linear or branched C1-C6 alkylene group, more preferably a C1-C4 alkylene group, and particularly a C3 alkylene, i.e. propyl, group.

Examples of suitable silane coupling agents include methyl trimethoxysilane, glycidoxypropyl trimethoxysilane, methacryloxypropyltri-methoxysilane, vinyl triethoxysilane, phenyl alkoxysilanes such as phenyl trialkoxysilane and diphenyl dialkoxysilane, dialkyl dialkoxysilanes such as dimethyl dimethoxysilane and dimethyl diethoxysilane, quaternary silanes, and amino silanes.

Amino silanes are preferred, and suitable materials include aminoethyl trimethoxysilane, aminoethyl triethoxysilane, aminopropyl trimethoxysilane, aminopropyl triethoxysilane, methylaminopropyl trimethoxysilane, ethylaminopropyl trimethoxysilane, aminopropyl tripropoxysilane, aminoisobutyl trimethoxysilane, and aminobutyl triethoxysilane. An especially preferred amino silane is aminopropyl triethoxysilane (NH₂—CH₂CH₂CH₂—Si—[OCH₂CH₃]₃).

The amount of silane coupling agent, or reaction product thereof, present in the coating layer is suitably up to 15%, preferably in the range from 1 to 10%, more preferably 3 to 7%, particularly 3.5 to 5%, and especially 4 to 4.5% by weight of based on the weight of the titanium dioxide core particles.

The silane coupling agent is suitably used in the coating layer in combination with an inorganic material and/or a fatty acid, both as defined herein. The inorganic material is suitably silica, is preferably amorphous, and more preferably is in a highly hydrated form, i.e. contains a high proportion of hydroxyl groups. The silica is preferably not in the form of dense silica. The fatty acid is preferably a stearic acid and/or salt thereof.

Suitably, titanium dioxide core particles are coated with inorganic material, preferably silica, are dispersed in water and heated to a temperature in the range from 50 to 80° C., after which the silane coupling agent is added which reacts with the surface of the inorganic material and/or the surface of the titanium dioxide core particles. The fatty acid and/or salt thereof is preferably applied after the inorganic material and the silane coupling agent.

The titanium dioxide particles may be coated prior to, or after any calcination stage. In a preferred embodiment, any coating is applied to the particles after any calcination stage. Thus, it is preferred that uncoated precursor titanium dioxide particles are subjected to the calcination process herein described.

In one embodiment, the titanium dioxide particles are coated in-situ, during the formation of a dispersion according to the present invention. Such coating may be applied by adding coating materials to the dispersion mixture before the milling process as herein described. Examples of materials which are suitable for the in-situ coating process are isostearic acid, oleth-3 phosphate, octyl/decyl phosphate, cetoleth-5 phosphate, PPG-5-ceteth-10 phosphate, trideceth-5 phosphate, dobanol C12-C15 phosphate, C9-C15 alkyl phosphate, glyceryl triacetate, sorbitan laurate, sorbitan isostearate, sodium lauryl sulfate, sodium methyl cocoyl taurate, and mixtures thereof.

The titanium dioxide, suitably coated, particles according to the present invention may have a BET specific surface area, measured as herein described, in the range from 15 to 43, suitably 20 to 38, preferably 24 to 34 more preferably 26 to 32, particularly 27 to 31, and especially 28 to 30 m²g⁻¹. The BET specific surface area may be reduced on coating the, preferably calcined, titanium dioxide particles by an amount in the range from 1.0 to 7.0, suitably 2.0 to 6.0, preferably 2.5 to 5.5, more preferably 3.0 to 5.0, particularly 3.5 to 4.5, and especially 3.8 to 4.2 m²g⁻¹.

The titanium dioxide, suitably coated, particles may have (i) an average pore diameter, measured as herein described by mercury porosimetry, in the range from 65 to 150, suitably 75 to 140, preferably 85 to 130, more preferably 95 to 120, particularly 100 to 115, and especially 105 to 110 nm; and/or (ii) a total pore area at 59,950.54 psia, measured as herein described by mercury porosimetry, in the range from 22 to 55, suitably 26 to 50, preferably 30 to 46, more preferably 33 to 43, particularly 35 to 41, and especially 37 to 39 m²g⁻¹.

The titanium dioxide, suitably coated, particles may have a mean aspect ratio d₁:d₂ (where d₁ and d₂, respectively, are the length and width of the particle) in the range from 1.05 to 1.55:1, preferably 1.10 to 1.50:1, more preferably 1.15 to 1.45:1, particularly 1.20 to 1.40:1, and especially 1.25 to 1.35:1. The third axis of the particles (or depth) is preferably approximately the same dimensions as the width.

The mean length by number of the titanium dioxide particles is suitably in the range from 32.0 to 56.0 nm, preferably 37.0 to 51.0 nm, more preferably 40.0 to 48.0 nm, particularly 42.0 to 46.0 nm, and especially 43.0 to 45.0 nm. The mean width by number of the particles is suitably in the range from 22.0 to 46.0 nm, preferably 27.0 to 41.0 nm, more preferably 30.0 to 38.0 nm, particularly 32.0 to 36.0 nm, and especially 33.0 to 35.0 nm. The size of the titanium dioxide particles can be determined, as herein described, by measuring the length and width of particles selected from a photographic image obtained by using a transmission electron microscope.

The titanium dioxide, suitably coated, particles may have a mean crystal size (measured by X-ray diffraction as herein described) in the range from 30.0 to 51.0 nm, suitably 34.0 to 51.0 nm, preferably 37.0 to 47.0 nm, more preferably 39.0 to 44.0 nm, particularly 41.0 to 44.0 nm, and especially 42.0 to 43.0 nm.

The size distribution of the crystal size of the titanium dioxide particles can be important, and suitably at least 50%, preferably at least 60%, more preferably at least 70%, particularly at least 80%, and especially at least 90% by weight of the titanium dioxide particles have a crystal size within one or more of the above preferred ranges for the mean crystal size.

The size of the titanium dioxide, suitably coated, particles can be determined, as herein described, by measuring the length and width of particles selected from a photographic image obtained by using a transmission electron microscope.

The titanium dioxide particles according to the present invention may be in the form of a free-flowing powder. A powder having the required particle size may be produced by milling processes known in the art. The final milling stage of the titanium dioxide is suitably carried out in dry, gas-borne conditions to reduce aggregation. A fluid energy mill can be used in which the aggregated titanium dioxide powder is continuously injected into highly turbulent conditions in a confined chamber where multiple, high energy collisions occur with the walls of the chamber and/or between the aggregates. The milled powder is then carried into a cyclone and/or bag filter for recovery. The fluid used in the energy mill may be any gas, cold or heated, or superheated dry steam.

The titanium dioxide particles may be formed into a slurry, or preferably a liquid dispersion, in any suitable aqueous or organic liquid medium. By liquid is meant liquid at ambient temperature (e.g. at 25° C.), and by dispersion is meant a true dispersion, i.e. where the solid particles are stable to aggregation. The particles in the dispersion are relatively uniformly dispersed and resistant to settling out on standing, but if some settling out does occur, the particles can be easily re-dispersed by simple agitation.

Alternatively, the titanium dioxide particles may be in the form of a lotion or cream of a solid and/or semi-solid dispersion. Suitable solid or semi-solid dispersions may contain, for example, in the range from 50 to 90%, preferably 60 to 85% by weight of titanium dioxide particles, together with any one or more of the liquid media disclosed herein, or a high molecular weight polymeric material, such as a wax, e.g. glyceryl monostearate.

For use in a sunscreen product, cosmetically acceptable materials are preferred as the liquid medium. The liquid medium may be water, or an organic medium such as a liquid, e.g. vegetable, oil, fatty acid glyceride, fatty acid ester and/or fatty alcohol. One suitable organic medium is a siloxane fluid, especially a cyclic oligomeric dialkylsiloxane, such as the cyclic pentamer of dimethylsiloxane known as cyclomethicone. Alternative fluids include dimethylsiloxane linear oligomers or polymers having a suitable fluidity and phenyltris(trimethylsiloxy)silane (also known as phenyltrimethicone).

Examples of other suitable organic media include non-polar materials such as C13-C14 isoparaffin, isohexadecane, paraffinum liquidum (mineral oil), squalane, squalene, hydrogenated polyisobutene, and polydecene; and polar materials such as C12-C15 alkyl benzoate, caprylic/capric triglyceride, cetearyl isononanoate, ethylhexyl isostearate, ethylhexyl palmitate, isononyl isononanoate, isopropyl isostearate, isopropyl myristate, isostearyl isostearate, isostearyl neopentanoate, octyldodecanol, pentaerythrityl tetraisostearate, PPG-15 stearyl ether, triethylhexyl triglyceride, dicaprylyl carbonate, ethylhexyl stearate, helianthus annus (sunflower) seed oil, isopropyl palmitate, and octyldodecyl neopentanoate, triethylhexanoin, ethylhexyl cocoate, propylene glycol isostearate, glyceryl isostearate, triisostearin, diethoxyethyl succinate, caprylyl eicosanoate, ethylhexyl hydroxystearate, lauryl lactate, butyl stearate, diisobutyl adipate, diisopropyl adipate, ethyl oleate, isocetyl stearate, propylene glycol dicaprylate/dicaprate, pentaerythrityl tetracaprylate/tetracaprate, oleyl oleate, propylene glycol isoceteth-3 acetate, PPG-3 benzyl ether myristate, cetearyl ethylhexanoate, ethylhexyl pelargonate, PPG-2 myristyl ether propionate, C14-18 alkyl ethylhexanoate, and mixtures thereof.

In one embodiment the organic medium is selected from the group consisting of isostearyl isostearate, isopropyl isostearate, triisostearin, ethyl oleate, dicaprylyl ether, and mixtures thereof.

In one embodiment the organic medium is a plant oil, such as those selected from the group consisting of sweet almond oil, olive oil, avocado oil, grapeseed oil, sunflower oil, meadowfoam seed oil, carrot oil, and mixtures thereof.

The dispersion according to the present invention may also contain a dispersing agent in order to improve the properties thereof. The dispersing agent is suitably present in the range from 1 to 30%, preferably 4 to 20%, more preferably 6 to 15%, particularly 8 to 12%, and especially 9 to 11% by weight based on the total weight of titanium dioxide particles.

Suitable dispersing agents include substituted carboxylic acids, soap bases and polyhydroxy acids. Typically the dispersing agent can be one having a formula R.CO.AX in which A is a divalent atom such as O, or a divalent bridging group. X can be hydrogen or a metal cation, or a primary, secondary or tertiary amino group or a salt thereof with an acid or a quaternary ammonium salt group. R may be the residue of a polyester chain which together with the —CO— group is derived from a hydroxy carboxylic acid of the formula HO—R′—COOH. As examples of typical dispersing agents are those based on ricinoleic acid, hydroxystearic acid, hydrogenated castor oil fatty acid which contains in addition to 12-hydroxystearic acid small amounts of stearic acid and palmitic acid. Dispersing agents based on one or more polyesters or salts of a hydroxycarboxylic acid and a carboxylic acid free of hydroxy groups can also be used. Compounds of various molecular weights can be used. Polyglyceryl-3 polyricinoleate and polyhydroxystearic acid are preferred dispersing agents. Polyglyceryl-3 polyricinoleate is particularly preferred when the coating layer of titanium dioxide particles comprises a silane coupling agent as herein defined. Polyhydroxystearic acid is particularly preferred when the coating layer of titanium dioxide particles does not comprise a silane coupling agent.

Other suitable dispersing agents are those monoesters of fatty acid alkanolamides and carboxylic acids and their salts. Suitable alkanolamides, for example, include those based on ethanolamine, propanolamine or aminoethyl ethanolamine. The dispersing agent can be one of those commercially referred to as a hyper dispersant. Polyhydroxystearic acid is a particularly preferred dispersing agent in organic media.

Suitable dispersing agents for use in an aqueous medium include a polymeric acrylic acid or a salt thereof. Partially or fully neutralized salts are usable e.g. the alkali metal salts and ammonium salts. Examples of dispersing agents are polyacrylic acids, substituted acrylic acid polymers, acrylic copolymers, sodium and/or ammonium salts of polyacrylic acids and sodium and/or ammonium salts of acrylic copolymers. Such dispersing agents are typified by polyacrylic acid itself and sodium or ammonium salts thereof as well as copolymers of an acrylic acid with other suitable monomers such as a sulphonic acid derivative such as 2-acrylamido 2-methyl propane sulphonic acid. Comonomers polymerisable with the acrylic or a substituted acrylic acid can also be one containing a carboxyl grouping. Usually the dispersing agents for use in an aqueous medium have a molecular weight in the range from 1,000 to 10,000 and are preferably substantially linear molecules. Materials such as sodium citrate may also be used as a co-dispersant.

An advantage of the present invention is that dispersions, particularly liquid, can be produced which suitably contain at least 30%, preferably at least 40%, more preferably at least 45%, particularly at least 50%, especially at least 55%, and generally up to 65%, by weight of titanium dioxide particles based on the total weight of the dispersion.

In one embodiment, the titanium dioxide particles according to the present invention, suitably calcined, have a volume based median particle diameter (equivalent spherical diameter corresponding to 50% of the volume of all the particles, read on the cumulative distribution curve relating volume (mass) % to the diameter of the particles—often referred to as the “D(v,0.5)” value)) in dispersion, measured as herein described, of (i) greater than 175 nm, suitably greater than 180 nm, more suitably greater than 200 nm, even more suitably greater than 220 nm, preferably greater than 235 nm, more preferably greater than 245 nm, particularly greater than 255 nm, and especially greater than 265 nm; and/or (ii) less than 360 nm, suitably less than 340 nm, more suitably less than 320 nm, preferably less than 305 nm, more preferably less than 295 nm, particularly less than 285 nm, and especially less than 275 nm; and/or (iii) any combination of (i) and (ii).

In one embodiment, the titanium dioxide particles, suitably calcined, have a D(v,0.5) value of greater than 175 nm, preferably in the range from 180 to 230 nm, more preferably 185 to 210, particularly 190 to 200, and especially 193 to 197 nm.

The size distribution of the titanium dioxide particles can also be an important parameter in obtaining the required properties. In one embodiment, (i) less than 10% by volume of titanium dioxide particles have a volume based diameter of more than 50 nm, suitably more than 45 nm, more suitably more than 40 nm, preferably more than 35 nm, more preferably more than 32 nm, particularly more than 28 nm, and especially more than 25 nm below the volume based median particle diameter; and/or (ii) less than 16% by volume of titanium dioxide particles have a volume based diameter of more than 45 nm, suitably more than 40 nm, more suitably more than 35 nm, preferably more than 30 nm, more preferably more than 25 nm, particularly more than 20 nm, and especially more than 18 nm below the volume based median particle diameter; and/or (iii) more than 90% by volume of titanium dioxide particles have a volume based diameter of less than 140 nm, suitably less than 125 nm, more suitably less than 115 nm, preferably less than 105 nm, more preferably less than 95 nm, particularly less than 85 nm, and especially less than 80 nm above the volume based median particle diameter; and/or (iv) more than 84% by volume of titanium dioxide particles have a volume based diameter of less than 100 nm, suitably less than 85 nm, more suitably less than 75 nm, preferably less than 65 nm, more preferably less than 55 nm, particularly less than 45 nm, and especially less than 40 nm above the volume based median particle diameter; and/or (v) any combination of (i), (ii), (iii) and/or (iv).

In one embodiment, the titanium dioxide particles according to the present invention, suitably calcined, have a number based median particle diameter (equivalent spherical diameter corresponding to 50% of the number of all the particles, read on the cumulative distribution curve relating volume % to the diameter of the particles—often referred to as the “D(n,0.5)” value)) in dispersion, measured as herein described, of (i) greater than 120 nm, suitably greater than 135 nm, more suitably greater than 145 nm, even more suitably greater than 155 nm, preferably greater than 165 nm, more preferably greater than 175 nm, particularly greater than 185 nm, and especially greater than 195 nm; and/or (ii) less than 265 nm, suitably less than 255 nm, more suitably less than 245 nm, preferably less than 235 nm, more preferably less than 225 nm, particularly less than 215 nm, and especially less than 205 nm; and/or (iii) any combination of (i) and (ii).

In one embodiment, the titanium dioxide particles, suitably calcined, have a D(n,0.5) value of greater than 100 nm, preferably in the range from 110 to 175 nm, more preferably 120 to 155, particularly 130 to 145, and especially 135 to 140 nm.

In one embodiment, (i) less than 10% by number of titanium dioxide particles have a number based diameter of more than 50 nm, suitably more than 45 nm, more suitably more than 40 nm, preferably more than 35 nm, more preferably more than 32 nm, particularly more than 28 nm, and especially more than 25 nm below the number based median particle diameter; and/or (ii) less than 16% by number of titanium dioxide particles have a number based diameter of more than 45 nm, suitably more than 40 nm, more suitably more than 35 nm, preferably more than 30 nm, more preferably more than 25 nm, particularly more than 20 nm, and especially more than 18 nm below the number based median particle diameter; and/or (iii) more than 90% by number of titanium dioxide particles have a number based diameter of less than 100 nm, suitably less than 85 nm, more suitably less than 70 nm, preferably less than 60 nm, more preferably less than 50 nm, particularly less than 45 nm, and especially less than 40 nm above the number based median particle diameter; and/or (iv) more than 84% by number of titanium dioxide particles have a number based diameter of less than 85 nm, suitably less than 70 nm, more suitably less than 55 nm, preferably less than 45 nm, more preferably less than 35 nm, particularly less than 30 nm, and especially less than 25 nm above the number based median particle diameter; and/or (v) any combination of (i), (ii), (iii) and/or (iv).

The size of the titanium dioxide particles in dispersion according to the present invention may be measured by techniques based on sedimentation analysis. The volume based median particle diameter may be determined by plotting a cumulative distribution curve representing the percentage of particle volume below chosen particle sizes and measuring the 50th percentile. The number based median particle diameter may be determined by plotting a cumulative distribution curve representing the percentage of particle numbers below chosen particle sizes and measuring the 50th percentile. The median particle volume and number diameter and particle size distribution thereof of the titanium dioxide particles is suitably measured by forming a dispersion of titanium dioxide particles and using a Brookhaven particle sizer, as herein described.

The size of the titanium dioxide particles in dispersion according to the present invention may also be measured by techniques based on light scattering. The intensity of scattered light is measured, where this function is fit to obtain a size, using algorithms which determine (i) the cumulant (or Z-average) mean particle size, giving one overall average particle size, and (ii) the peak size which gives a mean size based on the intensity of the scattered light. Intensity values can be converted to a number or volume distribution using Mie theory. This distribution describes the relative proportion of multiple components in the sample based on their mass or volume rather than based on their scattering (Intensity).

In one embodiment the titanium dioxide particles in dispersion have a Z-average particle size, measured by light scattering as herein described, of (i) greater than 80 nm, suitably greater than 100 nm, more suitably greater than 115 nm, preferably greater than 125 nm, more preferably greater than 130 nm, particularly greater than 135 nm, and especially greater than 140 nm; and/or (ii) less than 230 nm, suitably less than 200 nm, more suitably less than 185 nm, even more suitably less than 170 nm, preferably less than 165 nm, more preferably less than 160 nm, particularly less than 155 nm, and especially less than 150 nm; and/or (iii) any combination of (i) and (ii).

In one embodiment, the titanium dioxide particles in dispersion have a Z-average particle size in the range from 135 to 230 nm, preferably 155 to 210 nm, more preferably 165 to 200, particularly 175 to 190, and especially 180 to 185 nm.

In one embodiment the titanium dioxide particles in dispersion have an intensity mean particle size, measured by light scattering as herein described, of (i) greater than 90 nm, suitably greater than 110 nm, more suitably greater than 125 nm, preferably greater than 135 nm, more preferably greater than 145 nm, particularly greater than 150 nm, and especially greater than 155 nm; and/or (ii) less than or equal to 250 nm, suitably less than 220 nm, more suitably less than 200 nm, even more suitably less than 185 nm, preferably less than 175 nm, more preferably less than 170 nm, particularly less than 165 nm, and especially less than 160 nm; and/or (iii) any combination of (i) and (ii).

In one embodiment, the titanium dioxide particles in dispersion have an intensity mean particle size in the range from 150 to 250 nm, preferably 175 to 230 nm, more preferably 185 to 220, particularly 195 to 210, and especially 200 to 205 nm.

The titanium dioxide particles according to the present invention preferably exhibit acceptable transparency, and may have an extinction coefficient at 524 nm (E₅₂₄), measured as herein described, of (i) less than or equal to 7.5, suitably less than or equal to 7.0, preferably less than or equal to 6.8, more preferably less than or equal to 6.6, particularly less than or equal to 6.5, and especially less than or equal to 6.45 l/g/cm; and/or (ii) greater than or equal to 4.7, suitably greater than or equal to 5.2, more suitably greater than or equal to 5.7, preferably greater than or equal to 6.0, more preferably greater than or equal to 6.2, particularly greater than or equal to 6.3, and especially greater than or equal to 6.35 l/g/cm; and/or (iii) any combination of (i) and (ii).

The titanium dioxide particles exhibit effective UV absorption, and may have (i) an extinction coefficient at 360 nm (E₃₆₀), measured as herein described, of greater than 20, suitably greater than 27, more suitably in the range from 32 to 50, preferably 36 to 46, more preferably 39 to 44, particularly 40 to 43, and especially 41 to 42 l/g/cm; and/or (ii) an extinction coefficient at 308 nm (E₃₀₈), measured as herein described, of greater than 45, suitably greater than 50, more suitably in the range from 55 to 76, preferably 59 to 73, more preferably 62 to 70, particularly 64 to 68, and especially 65 to 67 I/g/cm, and/or (iii) any combination of (i), and (ii).

In one embodiment, the titanium dioxide particles may have an E₃₀₈×E₃₆₀ value of (i) less than 3500, suitably less than or equal to 3300, preferably less than or equal to 3150, more preferably less than or equal to 2950, particularly less than or equal to 2850, and especially less than or equal to 2800 (l/g/cm)²; and/or (ii) greater than 1800, suitably greater than 2100, more suitably greater than or equal to 2300, preferably greater than or equal to 2450, more preferably greater than or equal to 2550, particularly greater than or equal to 2650, and especially greater than or equal to 2700 (l/g/cm)²; and/or (iii) any combination of (i) and (ii).

In one embodiment, the titanium dioxide particles may have an (E₃₀₈×E₃₆₀)/E₅₂₄ value of (i) greater than 300, suitably greater than or equal 320, more suitably greater than or equal to 340, preferably greater than or equal to 365, more preferably greater than or equal to 385, particularly greater than or equal to 405, and especially greater than or equal to 425 l/g/cm; and/or (ii) less than 650, suitably less than or equal to 570, preferably less than or equal to 520, more preferably less than or equal to 485, particularly less than or equal to 465, and especially less than or equal to 445 l/g/cm; and/or (iii) any combination of (i) and (ii).

In one embodiment, the titanium dioxide particles may have an E₅₂₄×E₃₆₀ value of (i) less than 350, suitably less than or equal to 320, preferably less than or equal to 300, more preferably less than or equal to 285, particularly less than or equal to 275, and especially less than or equal to 270 (l/g/cm)²; and/or (ii) greater than 190, suitably greater than or equal to 215, preferably greater than or equal to 230, more preferably greater than or equal to 245, particularly greater than or equal to 255, and especially greater than or equal to 260 (l/g/cm)²; and/or (iii) any combination of (i) and (ii).

The titanium dioxide particles may have λ(max), measured as herein described, in the range from 290 to 355, suitably 300 to 340, preferably 308 to 330, more preferably 313 to 324, particularly 316 to 320, and especially 317 to 319 nm.

In one embodiment, the titanium dioxide particles may have (i) an E₃₆₀/E₅₂₄ ratio of greater than 5.0, suitably in the range from 5.5 to 8.0, preferably 5.9 to 7.3, more preferably 6.2 to 6.9, particularly 6.4 to 6.7, and especially 6.5 to 6.6; and/or (ii) an E₃₀₈/E₅₂₄ ratio of greater than 5.0, suitably in the range from 7.0 to 15.0, preferably 8.0 to 13.0, more preferably 9.0 to 12.0, particularly 9.5 to 11.5, and especially 10.0 to 11.0; and/or (iii) an E₃₆₀/E₅₂₄ ratio×E₃₀₈/E₅₂₄ ratio of greater than 30, suitably in the range from 40 to 130, preferably 50 to 100, more preferably 55 to 85, particularly 60 to 75, and especially 65 to 70; and/or (iv) any combination of (i), (ii) and/or (iii).

The titanium dioxide particles may have an E₃₆₀/E₃₀₈ ratio in the range from 0.30 to 0.90, suitably to 0.40 to 0.85 preferably 0.45 to 0.80, more preferably 0.50 to 0.75, particularly 0.55 to 0.70, and especially 0.60 to 0.65.

The titanium dioxide particles when present, for example, in a 40% by weight dispersion suitably exhibit a change in whiteness ΔL, measured as herein described, of less than 50, preferably in the range from 10 to 40, more preferably 20 to 36, particularly 27 to 33, and especially 29 to 31.

A composition, preferably an end-use sunscreen product, containing the titanium dioxide particles according to the present invention preferably comprises greater than 0.5%, more preferably in the range from 1 to 25%, particularly 3 to 20%, and especially 5 to 15% by weight based on the total weight of the composition, of titanium dioxide particles herein described.

Such a composition according to the present invention suitably has (i) a Sun Protection Factor (SPF), measured as herein described, of greater than 10, preferably greater than 15, more preferably greater than 25, particularly greater than 35, and especially greater than 40, and generally up to 60, and/or (ii) a UVA Protection Factor (UVAPF) measured as herein described, of greater than 6, preferably greater than 8, more preferably greater than 10, particularly greater than 12, and especially greater than 13 and generally up to 20.

The composition suitably has a UVA/UVB ratio of less than 0.90, preferably in the range from 0.40 to 0.75, more preferably 0.50 to 0.70, particularly 0.60 to 0.66, and especially 0.62 to 0.64.

The composition suitably has a SPF/UVAPF ratio of less than 5, preferably in the range from 1.5 to 3.5, more preferably 2.2 to 3.2, particularly 2.5 to 2.9, and especially 2.6 to 2.8.

The critical wavelength of the composition suitably has a value greater than 360 nm, preferably in the range from 370 nm to 390 nm, more preferably 375 nm to 385 nm, particularly 377 nm to 381 nm, and especially 378 nm to 380 nm.

One surprising feature of the present invention is that the aforementioned SPF, UVAPF, and/or SPF/UVAPF ratio values can be obtained when the titanium dioxide herein described is essentially the only ultraviolet light attenuator present in the composition. By “essentially” is meant less than 3%, preferably less 2%, more preferably less than 1%, particularly less than 0.5%, and especially less than 0.1% by weight based on the total weight of the composition, of any other inorganic and/or organic UV absorber.

The titanium dioxide particles suitably exhibit a change in whiteness AL of a sunscreen product containing the particles, measured as herein described, of less than 20, preferably in the range from 5 to 16, particularly 10 to 15, and especially 13 to 14. The composition suitably has a ΔL/SPF ratio of less than 1, preferably in the range from 0.05 to 0.8, more preferably 0.2 to 0.6, particularly 0.3 to 0.5, and especially 0.35 to 0.45.

The titanium dioxide particles and dispersions of the present invention are useful as ingredients for preparing sunscreen compositions, especially in the form of oil-in-water or water-in-oil emulsions. The compositions may further contain conventional additives suitable for use in the intended application, such as conventional cosmetic ingredients used in sunscreens. As mentioned above, the particulate titanium dioxide as defined herein, may be the only ultra violet light attenuator present, but other sunscreening agents, such as other titanium dioxide, zinc oxide and/or other organic UV absorbers may also be added. For example, the titanium dioxide particles defined herein may be used in combination with other existing commercially available titanium dioxide and/or zinc oxide sunscreens.

The titanium dioxide particles and dispersions of the present invention may be used in combination with organic UV absorbers such as butyl methoxydibenzoylmethane (avobenzone), benzophenone-3 (oxybenzone), 4-methylbenzylidene camphor (enzacamene), benzophenone-4 (sulisobenzone), bis-ethylhexyloxyphenol methoxyphenyl triazine (bemotrizinol), diethylamino hydroxybenzoyl hexyl benzoate, diethylhexyl butamido triazone, disodium phenyl dibenzimidazole tetrasulfonate, drometrizole trisiloxane, ethylhexyl dimethyl PABA (padimate O), ethylhexyl methoxycinnamate (octinoxate), ethylhexyl salicylate (octisalate), ethylhexyl triazone, homosalate, isoamyl p-methoxycinnamate (amiloxate), isopropyl methoxycinnamate, menthyl anthranilate (meradimate), methylene bis-benzotriazolyl tetramethylbutylphenol (bisoctrizole), octocrylene, PABA (aminobenzoic acid), phenylbenzimidazole sulfonic acid (ensulizole), terephthalylidene dicamphor sulfonic acid, and mixtures thereof.

In this specification, the following test methods have been used:

1) Particle Size Measurement of Titanium Dioxide Particles

A small amount of titanium dioxide powder, typically 2 mg, was pressed into approximately 2 drops of ultra-pure water (ELGA Medica R7), for one or two minutes using the tip of a steel spatula. The resultant suspension was diluted with water and shaken vigorously. The sample was deposited on a carbon-coated grid suitable for transmission electron microscopy and air dried before loading onto a JOEL 2100F PE6-TEM. An accelerating voltage of 200 kV was used and images were taken at an appropriate, accurate magnification. About 300-500 particles were displayed at about 2 diameters spacing. A minimum number of 300 particles were sized using a transparent size grid consisting of a row of circles of gradually increasing diameter, representing spherical crystals. Under each circle a series of ellipsoid outlines were drawn representing spheroids of equal volume and gradually increasing eccentricity. The basic method assumes log normal distribution standard deviations in the 1.2-1.6 range (wider particle size distributions would require many more particles to be counted, for example of the order of 1000). The suspension method described above was suitable for producing almost totally separated titanium dioxide particles whilst introducing minimal crystal fracture. Any residual aggregates were sufficiently well defined that they, and any small debris, could be ignored, and effectively only individual particles included in the count. Mean length, mean width, mean aspect ratio and size distributions of the titanium dioxide particles were calculated from the above measurements.

2) Crystal Size Measurement of Titanium Dioxide Particles

Crystal size was measured by X-ray diffraction (XRD) line broadening. Diffraction patterns were measured using a Bruker D8 diffractometer equipped with an energy dispersive detector acting as a monochromator. The X-ray generator powder was set at 40 kV and 40 mA. Programmable slits of 0.6 mm were used to measure diffraction with a step size of 0.05°. The data was analysed by fitting the diffraction pattern between 22° and 48° 20 with a set of peaks corresponding to the reflection positions for rutile and, where anatase was present, an additional set of peaks corresponding to those reflections. The fitting process allowed for removal of the effects of instrument broadening on the diffraction line shapes. The value of mean crystal size was determined for the rutile 110 reflection (at approximately 27° 2θ) based on its full width at half maximum height (FWHM) using the Scherrer equation, described, e.g. in B. E. Warren, “X-Ray Diffraction”, Addison-Wesley, Reading, Massachusetts, 1969, pp 251-254.

3) Particle Median Diameter and Particle Size Distribution of Titanium Dioxide Particles in Dispersion

i) An organic liquid dispersion of titanium dioxide particles was produced by mixing 5 g of polyhydroxystearic acid (or polyglyceryl-3 polyricinoleate when a silane coupling agent is present in the coating layer) with 45 g of C12-C15 alkylbenzoate, and then adding 50 g of titanium dioxide powder into the mixture. The mixture was passed through a horizontal bead mill, operating at 4,500 r.p.m. and containing zirconia beads as grinding media, for 60 minutes. The dispersion of titanium dioxide particles was;

(a) diluted to between 15 and 25 g/l by mixing with isopropyl myristate. The diluted sample was analysed on a Brookhaven BI-XDC particle sizer in centrifugation mode, and the volume and number based median diameter, and particle size distributions measured. Measurements were performed at a speed of 1,000 rpm, and the particle sizes determined based on the time the particle takes to sediment in the detector according to Stokes law (determined using X-ray light); and/or

(b) diluted to between 1 and 10 g/L by mixing with a solution of C12-C15 alkylbenzoate containing 3% weight by weight polyhydroxystearic acid. The diluted sample was transferred to a disposable plastic cuvette and analysed on a Malvern Zetasizer Nano ZS. The instrument initially starts by measuring an equilibrium stage, followed by analyzing the scattered light intensity from the sample, determining hydrodynamic volume of the particles based on their Brownian motion in suspension. The cumulant mean (Z-average) value was calculated by the methods of cumulants described in, e.g. Koppel, D. E. “Analysis of Macromolecular Polydispersity in Intensity Correlation Spectroscopy: The Method of Cumulants” J. Chem. Phys 57 (11), pp 4814-4820, 1972. The intensity based mean diameter, number based mean diameter and volume (mass) based mean diameter were also determined.

ii) An aqueous dispersion was produced by mixing 6.2 g polyglyceryl-2 caprate, 2.6 g sucrose stearate, 2 g jojoba oil, 0.6 g squalane, 1 g caprylyl caprylate, 37.4 g of demineralised water, and then adding 50 g of titanium dioxide powder into the mixture. The mixture was passed through a horizontal bead mill, operating at 4,500 r.p.m. and containing zirconia beads as grinding media, for 60 minutes. The dispersion of titanium dioxide particles was;

(a) diluted to between 15 and 25 g/l by mixing with a 0.1% by weight aqueous solution of isodeceth-6). The diluted sample was analysed on a Brookhaven BI-XDC particle sizer in centrifugation mode, and the volume based median diameter, number based median diameter, and particle size distributions measured as described above for an organic dispersion; and/or

(b) diluted to between 1 and 10 g/L by mixing with demineralised water. The diluted sample was transferred to a disposable plastic cuvette and analysed on a Malvern Zetasizer Nano ZS. The instrument will initially start by measuring an equilibrium stage, followed by analyzing the scattered light intensity from the sample, determining hydrodynamic volume of the particles based on their Brownian motion in suspension. The cumulant mean (Z-average) value, intensity based mean diameter, number based mean diameter and volume (mass) based mean diameter were measured as described above for an organic dispersion.

4) BET Specific Surface Area of Titanium Dioxide Particles

The BET specific surface area was measured using a Micromeritics Gemini VII 2390P. 0.4-0.5 g of dry titanium dioxide powder was introduced into sample tubes, degassed for 10 minutes under nitrogen at room temperature, before being heated to 200° C. and held at this temperature for 3 hours, again under nitrogen. The dry sample was immersed in liquid nitrogen (−196° C.) and once the sample was frozen, the specific surface area (SSA) was analysed using nitrogen.

5) Mercury Porosimetry Pore Size of Titanium Dioxide Particles

The pore size distribution was measured using a Micromeritics Autopore V Porosimeter. Approximately 0.1 g of dry titanium dioxide powder was weighed into the bulb of the penetrometer. The penetrometer containing the titanium dioxide was loaded into the Micromeritics Autopore V porosimeter and measurements were carried out between 0.33 to 60,000 psia during intrusion and extrusion cycles. The average pore diameter and total pore area at 59,950.54 psia were determined.

6) Change in Whiteness of Titanium Dioxide Particles

i) An organic or aqueous titanium dioxide dispersion (e.g. as described in 3) above) was coated on to the surface of a glossy black card and drawn down using a No 2 K bar to form a film of 12 microns wet thickness. The film was allowed to dry at room temperature for 10 minutes and the whiteness of the coating on the black surface (L_{F}) measured using a Minolta CR300 colorimeter. The change in whiteness ΔL was calculated by subtracting the whiteness of the substrate (L_{S}) from the whiteness of the coating (L_{F}).

ii) A sunscreen formulation (e.g. as described in Example 3) was coated on to the surface of a glossy black card and drawn down using a No 2 K bar to form a film of 12 microns wet thickness. The film was allowed to dry at room temperature for 10 minutes and the whiteness of the coating on the black surface (L_{F}) measured using a Minolta CR300 colorimeter. The change in whiteness ΔL was calculated by subtracting the whiteness of the substrate (L_{S}) from the whiteness of the coating (L_{F}).

7) Sun Protection Factor and UVA/UVB Ratio

The Sun Protection Factor (SPF) of a sunscreen formulation (e.g. as described in Example 3) was determined using the in vitro method of Diffey and Robson, J. Soc. Cosmet. Chem. Vol. 40, pp 127-133,1989. This method was also used to identify the UVA/UVB ratio of the sunscreen formulation, determined by analysing the area under the absorption curve related to the UVB portion of the curve divided by the area related to the UVA portion of the curve.

8) UVA Protection Factors and Critical Wavelength

The UVA Protection Factors (UVAPF₀ and UVAPF) of a sunscreen formulation (e.g. as described in Example 3) were determined as described in COLIPA Guidelines ‘Method for In Vitro Determination of UVA Protection Provided by Sunscreen Products Edition of 2011’. A Labsphere UV-2000S UV transmittance analyzer was used. This method was also used to determine the critical wavelength of the formulation which indicates the wavelength below which 90% of the area under the absorbance curve resides.

A blank (100% transmission) sample was produced by spreading 1.30 mg cm⁻² (equivalent to 0.0325 g) of glycerine onto the roughened surface of a polymethyl methacrylate (PMMA) plate (Helioplates HD6, ex Laboratoire Helios Science Cosmetique). The sunscreen formulation was applied to the roughened surface of an identical PMMA plate, at a concentration of 1.30 mg cm⁻² (equivalent to 0.0325 g) as a series of small dots distributed evenly across the surface of the plate. Immediately after applying, the formulation was spread over the whole surface of the plate using a latex gloved finger. The coated plate was left to dry in the dark for 15 minutes. Immediately after drying, a total of 9 UV transmission spectra (290 to 400 nm) were recorded for each plate at different locations. Three different plates were used to give an average of 27 readings of the UV transmission data at each wavelength. The UV-radiation transmitted through the coated plates at each 1 nm increment was quantified. The individual transmission measurements obtained at each wavelength increment were used to calculate an initial UVA protection factor (UVAPF₀). Using a long-arc xenon Atlas Suntest CPS+ insolator, the same sunscreen formulation treated plate was then exposed to a single UV dose of simulated sun exposure, which was calculated by the instrument and related to the UVAPF₀, after which a second series of transmission measurements were made through the sample. The same number of measurements (i.e. 9×3 plates) were taken prior to the simulated sun exposure. Again, the transmission values were converted to absorbance values and a post exposure UVA protection factor (UVAPF) was calculated.

9) Extinction Coefficients

i) 0.1 g sample of an organic liquid titanium dioxide dispersion (e.g. as described in 3) above) was diluted with 100 ml of cyclohexane. This diluted sample was then further diluted with cyclohexane in the ratio sample:cyclohexane of 1:19. The total dilution was 1:20,000.

ii) 0.1 g sample of an aqueous titanium dioxide dispersion (e.g. as described in 3) above) was diluted with 100 ml of demineralised water. This diluted sample was then further diluted with demineralised water in the ratio sample:demineralised water of 1:19. The total dilution was 1:20,000.

The diluted samples produced in i) and/or ii) were placed in a spectrophotometer (Perkin-Elmer Lambda 650 UV/VIS Spectrophotometer) with a 1 cm path length and the absorbance, of UV and visible light measured. Extinction coefficients were calculated from the equation A=E.c.l, where A=absorbance, E=extinction coefficient in litres per gram per cm, c=concentration in grams per litre, and l=path length in cm.

The invention is illustrated by the following non-limiting examples.

### EXAMPLES

### Example 1

1 mole of titanium oxydichloride in acidic solution was reacted with 3 moles of NaOH in aqueous solution. After the initial reaction period, the temperature was increased to above 70° C., and stirring continued. The reaction mixture was neutralised by the addition of aqueous NaOH, and allowed to cool below 70° C. After filtering, the resulting filter cake of precursor titanium dioxide particles was further dried using a rotary dryer operating at 6 r.p.m, to 20% by weight of water. Using a screw conveyor, this material was fed into a rotating calciner operating at 710° C. with a residence time of 20 minutes. The processed titanium dioxide was ground into a fine powder using an IKA Werke dry powder mill operating at 3,250 r.p.m. The powder was re-slurried in demineralised water. To the resulting slurry, an alkaline solution of sodium aluminate was added, equivalent to 3.5% by weight Al₂O₃ on TiO₂ weight, whilst keeping the pH below 11. The temperature was maintained below 60° C. during the addition. The temperature of the slurry was then increased to 75° C., and 4.6% by weight of sodium stearate on TiO₂ dissolved in hot water was added. The slurry was equilibrated for 45 minutes and neutralised by adding 20% hydrochloric acid dropwise over 15 minutes, before the slurry was allowed to cool to less than 50° C. The slurry was filtered using a Buchner filter until the cake conductivity at 100 gdm⁻³ in water was <150 μS. The filter cake was oven-dried for 24 hours at 110° C. and ground into a fine powder by an IKA Werke dry powder mill operating at 3,250 r.p.m.

A dispersion was produced by mixing 5.5 g of polyhydroxystearic acid with 39.5 g of C12-C15 alkylbenzoate, and then adding 55 g of dried calcined titanium dioxide powder produced above, into the mixture. The mixture was passed through a horizontal bead mill, operating at 4,500 r.p.m. and containing zirconia beads as grinding media, for 60 minutes.

The precursor titanium dioxide particles, calcined titanium dioxide particles, coated titanium dioxide particles and dispersion thereof, were subjected to the test procedures herein described, and exhibited the following properties;

1) Precursor Titanium Dioxide Particles:

BET specific surface area=101 m²g⁻¹

Mercury porosimetry average pore diameter=77.6 nm

Mercury porosimetry total pore area at 59,950.54 psia=72.6 m²g⁻¹

Mean crystal size=10 nm

Mean length=75 nm

Mean width=15 nm

Mean aspect ratio=5.0:1

2) Calcined Titanium Dioxide Particles:

BET specific surface area=31.9 m²g⁻¹

Mercury porosimetry average pore diameter=119 nm

Mercury porosimetry total pore area at 59,950.54 psia=36.7 m²g⁻¹

Mean crystal size=42.4 nm

Mean length=44 nm

Mean width=34 nm

Mean aspect ratio=1.3:1

3) Change in Titanium Dioxide Particle Properties on Calcining:

Reduction in BET specific surface area=68.4%

Increase in mercury porosimetry average pore diameter=53.4%

Reduction in mercury porosimetry total pore area at 59,950.54 psia=49.4%

Increase in mean crystal size=324%

Increase in mean width=126.7%

4) Coated Titanium Dioxide Particles:

BET specific surface area=28.6 m²g⁻¹.

Mercury porosimetry average pore diameter=107.9 nm

Mercury porosimetry total pore area at 59,950.54 psia=37.9 m²g⁻¹

5) Titanium Dioxide Dispersion:

(a) Particle size by sedimentation;

i) D (v,0.5)=271 nm,

ii) D (n,0.5)=200 nm,

(b) Particle size by light scattering

i) Z-average =148 nm

ii) Intensity mean =158 nm

(c) Extinction coefficients;

| E₅₂₄ | E₃₀₈ | E₃₆₀ | E (max) | λ (max) | E₃₀₈/E₅₂₄ | E₃₆₀/E₅₂₄ |
|---|---|---|---|---|---|---|
| 6.5 | 65.8 | 43.1 | 67.4 | 317 | 10.1 | 6.6 |

| E₃₆₀/E₃₀₈ | E₅₂₄ + E₃₆₀ | E₅₂₄ × E₃₆₀ | E₃₀₈ × E₃₆₀ | (E₃₀₈ × E₃₆₀)/E₅₂₄ |
|---|---|---|---|---|
| 0.66 | 49.6 | 280.2 | 2836 | 436.3 |

### Example 2

An aqueous dispersion was produced by mixing 6.2 g polyglyceryl-2 caprate, 2.6 g sucrose stearate, 2 g jojoba oil, 0.6 g squalane, 1 g caprylyl caprylate, 37.4 g of demineralised water, and then adding 50 g of titanium dioxide powder produced in Example 1. The mixture was passed through a horizontal bead mill, operating at 4,500 r.p.m. and containing zirconia beads as grinding media, for 60 minutes. The titanium dioxide dispersion was subjected to the test procedures herein described, and exhibited the following properties;

Extinction Coefficients;

| E₅₂₄ | E₃₀₈ | E₃₆₀ | E (max) | λ (max) | E₃₀₈/E₅₂₄ | E₃₆₀/E₅₂₄ |
|---|---|---|---|---|---|---|
| 7.3 | 65.7 | 45.4 | 68.0 | 319 | 9.0 | 6.2 |

| E₃₆₀/E₃₀₈ | E₅₂₄ + E₃₆₀ | E₅₂₄ × E₃₆₀ | E₃₀₈ × E₃₆₀ | (E₃₀₈ × E₃₆₀)/E₅₂₄ |
|---|---|---|---|---|
| 0.69 | 52.7 | 331.4 | 2983 | 408.6 |

### Example 3

The titanium dioxide dispersion produced in Examples 1 was used to prepare a sunscreen emulsion formulation having the following composition;

| | | |
|---|---|---|
| Trade Name | INCI | % w/w |

Phase A

| | | |
|---|---|---|
| Arlacel ™ 165 (ex | Glyceryl stearate (and) | 6.0 |
| Croda) | PEG-100 stearate | |
| Span ™ 60 (ex Croda) | Sorbitan stearate | 0.5 |
| Tween ™ 60 (ex | Polysorbate 60 | 2.7 |
| Croda) | | |
| Stearyl alcohol | Stearyl alcohol | 1.0 |
| Light mineral oil | Mineral oil | 7.8 |
| Crodamol ™ OP (ex | Ethylhexyl palmitate | 2.5 |
| Croda) | | |
| DC 200 350 cps | Dimethicone | 2.0 |
| Unimer U-15 (ex | VP/Eicosene copolymer | 1.0 |
| Induchem) | | |
| TiO₂ dispersion (55% | | 18.2 |
| solids) produced in | | |
| Example 1) | | |

Phase B

| | | |
|---|---|---|
| Water | Aqua | 53.2 |
| Keltrol RD | Xanthan gum | 0.1 |
| Propylene gylcol | Propylene glycol | 4.0 |

Phase C

| | | |
|---|---|---|
| Euxyl K 350 | Phenoxyethanol (and) | 1.0 |
| | methylparaben (and) | |
| | ethylparaben (and) | |
| | ethylhexylglycerin (and) | |
| | propylene glycol | |

Procedure
1. Keltrol RD was dispersed into water, and the remaining water Phase A ingredients added to the mixture, which was heated to 65-80° C.
2. The oil Phase B ingredients were combined and heated to 75-80° C.
3. The oil phase was added to the water phase with stirring.
4. The mixture was homogenised for 1 minute.
5. The resulting emulsion was cooled to room temperature with stirring, with the Phase C preservative being added below 40° C.

The sunscreen formulation was subjected to the test procedures herein described, and exhibited the following properties;

i) SPF=34

ii) UVA/UVB ratio=0.684

iii) UVAPF=13

iv) Critical Wavelength=379 nm

v) ΔL=13.5

vi) ΔL/SPF ratio=0.40

### Example 4

1 mole of titanium oxydichloride in acidic solution was reacted with 3 moles of NaOH in aqueous solution. After the initial reaction period, the temperature was increased to above 70° C., and stirring continued. The reaction mixture was neutralised by the addition of aqueous NaOH, and allowed to cool below 70° C. After filtering, the resulting filter cake of precursor titanium dioxide particles was further dried using a fluid bed (at approximately 150 degrees for 2 hours), to 5% by weight of water. Using a screw conveyor, this material was fed into a rotating calciner operating at 710° C. The processed titanium dioxide was ground into a fine powder using an IKA Werke dry powder mill operating at 3,250 r.p.m. The powder was re-slurried in demineralised water. To the resulting slurry, an alkaline solution of sodium aluminate was added, equivalent to 3.5% by weight Al₂O₃ on TiO₂ weight, whilst keeping the pH below 11. The temperature was maintained below 60° C. during the addition. The temperature of the slurry was then increased to 75° C., and 4.6% by weight of sodium stearate on TiO₂ dissolved in hot water was added. The slurry was equilibrated for 45 minutes and neutralised by adding 20% hydrochloric acid dropwise over 15 minutes, before the slurry was allowed to cool to less than 50° C. The slurry was filtered using a Buchner filter until the cake conductivity at 100 gdm⁻³ in water was <150 μS. The filter cake was oven-dried for 24 hours at 110° C. and ground into a fine powder by an IKA Werke dry powder mill operating at 3,250 r.p.m.

A dispersion was produced by mixing 5 g of polyhydroxystearic acid with 45 g of C12-C15 alkylbenzoate, and then adding 50 g of dried calcined titanium dioxide powder produced above, into the mixture. The mixture was passed through a horizontal bead mill, operating at 4,500 r.p.m. and containing zirconia beads as grinding media, for 60 minutes.

The titanium dioxide dispersion was subjected to the test procedures herein described, and exhibited the following properties;

(a) Particle size by sedimentation;

iii) D (v,0.5)=196 nm,

iv) D (n,0.5)=137 nm,

(b) Particle size by light scattering

i) Z-average=182 nm

ii) Intensity mean=203 nm

(c) Extinction coefficients;

| E₅₂₄ | E₃₀₈ | E₃₆₀ | E (max) | λ (max) | E₃₀₈/E₅₂₄ | E₃₆₀/E₅₂₄ |
|---|---|---|---|---|---|---|
| 6.0 | 57.1 | 36.8 | 59.7 | 317 | 9.5 | 6.1 |

| E₃₆₀/E₃₀₈ | E₅₂₄ + E₃₆₀ | E₅₂₄ × E₃₆₀ | E₃₀₈ × E₃₆₀ | (E₃₀₈ × E₃₆₀)/E₅₂₄ |
|---|---|---|---|---|
| 0.64 | 42.8 | 220.8 | 2101 | 350.2 |

The above examples illustrate the improved properties of titanium dioxide particles, method of producing thereof, titanium dioxide dispersion, and/or sunscreen product, according to the present invention.

1. Titanium dioxide particles comprising a volume based median particle diameter D(v,0.5) of greater than 175 nm and an (E₃₀₈×E₃₆₀)/E₅₂₄ value of greater than 300 l/g/cm.

2. The titanium dioxide according to claim 1 comprising:
(i) a number based median particle diameter D(n,0.5) of greater than 100 nm, and/or
(ii) a Z-average particle size of greater than 80 nm, and/or
(iii) an intensity mean particle size of greater than 90 nm.

3. The titanium dioxide according to claim 1 comprising a mean aspect ratio of 1.05 to 1.55:1.

4. Titanium dioxide particles comprising:
(i) a mean crystal size of 30.0 to 51.0 nm, and/or
(ii) a mean aspect ratio of 1.05 to 1.55:1.

5. The titanium dioxide according to claim 4 comprising a mean width of 22.0 to

46. 0 nm.

6. The titanium dioxide according to claim 1 comprising:
(i) a mean crystal size of 30.0 to 51.0 nm, and/or
(ii) a mean width of 22.0 to 46.0 nm.

7. The titanium dioxide according to claim 1 comprising an (E₃₀₈×E₃₆₀)/E₅₂₄ value of greater than or equal to 320 l/g/cm.

8. The titanium dioxide according to claim 1 comprising a mean crystal size of 37.0 to 47.0 nm.

9. The titanium dioxide according to claim 1 comprising a BET specific surface area of 15 to 43 m²g⁻¹.

10. The titanium dioxide according to claim 1 comprising:
(i) a mercury porosimetry total pore area at 59,950.54 psia of 22 to 55 m²g⁻¹, and/or
(ii) a mercury porosimetry average pore diameter of 65 to 150 nm.

11. The titanium dioxide according to claim 1 comprising an E₃₀₈×E₃₆₀ value of greater than 1800 (l/g/cm)² and less than 3500 (l/g/cm)².

12. Titanium dioxide particlescomprising:
(i) an (E₃₀₈×E₃₆₀)/E₅₂₄ value of greater than or equal to 320 l/g/cm, and optionally (ii) an E₅₂₄ of less than or equal to 7.5 l/g/cm, and/or an E₃₀₈×E₃₆₀ value of greater than 2100 (l/g/cm)².

13. The titanium dioxide according to claim 1 comprising an (E₃₀₈×E₃₆₀)/E₅₂₄ value of 320 to less than 650 l/g/cm.

14. The titanium dioxide according to claim 1 comprising at least one selected from the group consisting of:
(i) an E₅₂₄ of 5.2 to 7.5 l/g/cm,
(ii) an E₃₆₀ of 32 to 50 l/g/cm,
(iii) an E₃₀₈ of greater than 45 l/g/cm, and
(iv) an E₃₀₈×E₃₆₀ value of greater than 1800 to 3300 (l/g/cm)².

15. The titanium dioxide according to claim 14 comprising at least two selected from the group consisting of (i), (ii), (iii) and (iv).

16. The titanium dioxide according to claim 15 comprising all of (i), (ii), (iii) and (iv).

17. A dispersion comprising a dispersing medium and titanium dioxide particles as defined in claim 1.

18. A sunscreen product comprising titanium dioxide particles as defined in claim 1.

19. A method of producing titanium dioxide particles which comprises:
(i) forming precursor titanium dioxide particles having a mean aspect ratio of 3.0 to 7.0:1,
(ii) calcining the precursor particles to produce calcined titanium dioxide particles having a mean crystal size of 30.0 to 51.0 nm and/or a mean aspect ratio of 1.05 to 1.55:1, and optionally (iii) applying an inorganic and/or organic coating to the calcined titanium dioxide particles.

20. The method according to claim 19 wherein (i) the mean aspect ratio of the calcined particles is 1.15 to 1.45:1, and/or (ii) the mean crystal size of the calcined particles is 37.0 to 47.0 nm.

21. The method according to claim 19, wherein the calcined titanium dioxide particles comprise:
(i) an E₅₂₄ of less than or equal to 7.5 l/g/cm, and/or
(ii) an E₃₀₈×E₃₆₀ value of greater than 1800 (l/g/cm)².

22. The method according to claim 19, wherein the calcined titanium dioxide particles comprise at least one selected from the group consisting of:
(i) an E₅₂₄ of 4.7 to 7.5 l/g/cm,
(ii) an E₃₆₀ of 32 to 50 l/g/cm,
(iii) an E₃₀₈ of greater than 45 l/g/cm, and
(iv) an E₃₀₈×E₃₆₀ value of greater than 1800 to 3300 (l/g/cm)².

23. The method according to claim 19, wherein on calcining:
(i) the mean width of the titanium dioxide particles is increased by 60 to 200%, and/or
(ii) the BET specific surface area is reduced by 35 to 95%, and/or
(iii) the mean crystal size is increased by 200 to 400%.

24.-28. (canceled)
